(19) United States
(12) Patent Application Publication
(10) Pub. No.: US 2020/0215009 A1
(43) Pub. Date: Jul. 9, 2020

(54) Optimized Solution for the Treatment of Alzheimer's, Stroke, Cardiovascular and other Amyloid Related Diseases

(71) Applicant: Richard Postrel, Miami Beach, FL (US)

(72) Inventor: Richard Postrel, Miami Beach, FL (US)

(21) Appl. No.: 15/713,627

(22) Filed: Sep. 23, 2017

(51) Int. Cl. A61K 31/198; A61K 45/06

(52) U.S. Cl. CPC A61K 31/198; A61K 45/06

(57) ABSTRACT

This invention provides systems, methods and compositions for the prevention of diseases caused by amyloid based deposits of proteins. This invention teaches procedures and therapeutic models for controlling amyloids and related plaque deposits as applicable to common diseases including, but not limited to: Alzheimer's, cardiovascular, stroke, arterial sclerosis, etc. By addressing plaque development at its earliest stage involving protein-protein interaction, ongoing plaque growth is arrested or substantially decreased while the balance of plaque deposition/management for expulsion of existing plaques is switched to favor the body's natural aberrant protein removal mechanisms. The preferred embodiment implements a dual approach for: a) attacking protein dimerization and oligomerization that serves as the cornerstone of the disease, and b) rebalancing the body's natural processes to enhance removal of existing plaques through inflammation management in concert with cessation of ongoing plaque formation. In normal disease progression, the body's adaptive mechanisms amplify inflammation that interferes with plaque expulsion and exacerbates the problem through reactive oxygen and pro-inflammatory chemokine release. This imbalance increases severity as the plaques continue to grow and proliferate. However, stopping new plaque deposits while concurrently supporting the natural destruction of existing plaques reverses the disease processes and immediately improves patient's conditions. The invention also provides procedures and/or kits for effective analysis to achieve earliest detection for maximum effect.

This invention provides a solution for curing Alzheimer's, cardiovascular, stroke and other diseases caused by amyloid plaque deposits. A two pronged approach stops the formation of new plaques and augments the body's processes to increase plaque removal. Plaques and plaque precursor elements can be observed in many tissues. Amyloid plaques deposited in the brain tissue are a cause of dementia in the course of Alzheimer's disease.

Alzheimer's disease (AD) is the most common cause of dementia, affecting tens of millions of individuals worldwide. An associated disease, Hereditary Cystatin C Amyloid Angiopathy (HCCAA), also referred to as Hereditary Cerebral Hemorrhage with Amyloidosis-Icelandic type (HCHWA-I), which also leads to undesired plaques formation, is a rare autosomal dominant genetic disease of Icelandic heritage. HCCAA is caused by a single nucleotide mutation (SNP) where a T replaces A changing a CTA codon for leucine to the CAA codon for glutamine. This mutation (L68Q) results in the exchange of leucine for glutamine at amino acid 68 of the protein and changes the geometry and binding characteristics of the cystatin C expression product. The resultant disease is classified as a cerebral amyloid angiopathy with its primary pathology being manifested by deposition of mutant cystatin C protein as amyloid aggregates in the walls of cerebral arteries. This leads to fatal cerebral hemorrhages in young adults. The average age of mutation carriers is 30 years. Significant subsets of patients develop dementia at an early age without having history of clinical stroke with symptoms comparable to very early age Alzheimer Disease (AD). A pharmaceutical substance effective in treating cystatin C associated disease would satisfy a long felt need for persons with this mutation and for symptomatically similar individuals forming amyloid aggregates.

Amyloidoses may degrade functions including, but not limited to: cardiac, digestive, hepatic, renal, nervous, splenic, circulatory, pancreatic, lymphatic, etc. systems. Minor effects may be observed elsewhere, for example in integumentary, buccal, osteo, lipid tissues, etc., where amyloid deposits may be present and assayable, with minimal harm to the biopsied individual. Family history, tuberculosis, race, age, multiple myeloma, rheumatoid arthritis, dialysis, type II diabetes, and chronic inflammation are recognized correlates and/or risk factors relating to amyloid diseases.

All types of amyloidosis have soluble precursors that may be expressed and secreted at or near the site of plaque formation or may be synthesized remotely and transported as solubilized protein for systemic distribution and formation of amyloid deposits affecting diverse locations and multiple organs. The healthy body has various mechanisms to prevent and remove these spurious deposits.

Plaque disease presents when the precipitation and deposition overwhelm the removal mechanisms causing deleterious inflammation which impedes the resorption processes provoking increased size and number of plaques whose presence leads to the disease. The interplay participates in a positive feedback spiral effect: increasing inflammation, impairing resorption in a downward self-feeding spiral. Slowing deposit activity addresses the disease at one end. But rebalancing the immune system's inflammatory proclivities towards anti-inflammatory paths can reverse the spiral and allow reversal of the disease.

Additionally, the immune interplay in amyloid metabolism has been studied extensively in Alzheimer's disease where microglial activity (having a function in nervous tissue similar to the functions of macrophages) has received great attention. The immune response comprises a multi-faceted balancing operation including both detrimental actions that promote neurodegeneration as well as countering actions that promote neuronal survival and tissue repair. Normal interactions of macrophages (and microglia) with neighboring cells suppress immunologic inducing activities of these cells dampening responses to pro-inflammatory signals. In the CNS, a membrane glycoprotein, fractalkine is expressed on the neuronal cell surface and links with the chemokine receptor CXCR1 on the microglia. Similarly neurons express CD200 that links to the CD200 ligand, CD200L inhibiting production of pro-inflammatory mediators by the microglia.

Furthermore, microglia have been observed extending and retracting processes, possibly tunneling nanotube or f-actin like structures, into the adjacent tissue with a radius of about 80 μm. These structures serve to repair and remodel neuronal synapses as well as other functions and structures. However, when protein deposits interfere with either the dampening interplay of interactions between cells or the ability of microglia to reach and repair neighbor cells microglia proliferate, do not receive the dampening signal linkages, and provoke an imbalance in the immune system operations. One result is an increase in production and release of reactive oxygen species which reacts with the deposited proteins and impedes their reabsorption.

In normal healthy operation, microglia and macrophages participate in a coordinated response that mediates tissue repair. Anti-inflammatory cytokines, IL-4, IL-10, IL-13, and TGFβ are produced by both glia and neurons as part of the TH-2 anti-inflammatory pathways. These are in balance between TH-1 pathways with pro-inflammatory interleukins: IL-1, 11-6, IL-12, and γ-interferon. Slowing the formation and growth of amyloid deposits by suppressing or blocking protein-protein agglomerations leading to precipitation and deposit helps to restore the immune system balance. This rebalancing can be further augmented by suppressing pro-inflammatory activities through the associated delivery of anti-inflammatory substances in a combination therapy as discussed below.

Thus preferred embodiments of the invention feature a duplex system whereby plaque deposition is halted and plaque resorption is increased. This two pronged approach not only arrests disease progression but can reverse the disease processes and symptoms.

### Amyloid Precursor—Cystatin C

Cystatin C is a type 2 cystatin, that will bind to and inhibit cysteine proteases including plant derived papain and mammalian cathepsins B, H and L. It is a low molecular weight, ubiquitously expressed, secretory protein, which regulates bone reabsorption, neutrophil chemotaxis, and the inflammatory response. Exogenously added cystatin C enhances IFN-gamma-induced activation of NF-kappaB and increases mRNA levels for inducible NO synthase (iNOS) as well as levels of nitric oxide in the cell culture medium. Decreased endothelial nitric oxide (NO) is a common feature of aging and cerebrovascular disease.

The present invention preferentially provides methods for decreasing dimerization, proteinaceous deposits, including plaques, and treating Hereditary Cystatin C Amyloid Angiopathy (HCCAA) and/or Alzheimer's disease (AD), and methods for treating cerebral angiopathy, in an individual, the methods generally involving administering to an individual having AD a therapeutically effective amount of an agent that reduces cystatin C levels and/or dimerization activity.

HCCAA is a systemic disorder and cystatin C is not only deposited within the central nervous system of patients, but also in peripheral tissues such as in the dermis of skin where disease progression can be non-invasively monitored. Given similarities in the disease pathogenesis with AD, it is reasonable to hypothesize that therapy that would be effective in preventing HCCAA may also benefit patients with AD.

Excess cystatin C has been implicated in a plurality of diseases including, but not limited to: atherosclerosis, diabetes, obesity, coronary heart disease, stroke, cerebral hemorrhage, dementia, etc.

Cerebrovascular amyloid deposition (CAA) is a generic disease of unknown causes characterized by amyloid protein deposition in the blood vessels of the brain. Cystatin C has also been determined immunohistochemically to co-localized with Aβ in sporadic CAA and to co-immunoprecipitate with Aβ precursor protein.

Severe forms of CAA cause cerebrovascular disorders such as lobar cerebral hemorrhage and leukoencephalopathy. These may present with dementia similar to dementias seen in Alzheimer's disease (AD). Several cerebrovascular amyloid proteins have been characterized and include cystatin C, Aβ, cystatin C/Aβ, prion protein, variant transthyretins (ATTR) in meningovascular amyloidoses, mutated gelsolin (AGEI) in familial amyloidosis of Finnish type, disease associated prion protein (PrP(Sc)) in a variant of the Gerstmann-Straussler-Scheinker syndrome. Among the multiple presentations of CAA, the Aβ type is the most commonly found in elderly individuals and in patients with AD. Several mutations found in the genes encoding amyloid precursor proteins have been associated with hereditary CAA.

Apart from simply being co-precipitated with Aβ in the brain, there are studies showing that cystatin C can also polymerize and give rise to amyloid bodies. Especially in hereditary cerebral hemorrhage with amyloidosis Icelandic-type (HCHWA-I), an autosomal dominant disorder found in Icelanders, cystatin C is directly involved in the pathogenesis of CAA. This disorder is associated with the L68Q (68Leu→Gln) mutation and a frequent absence of ten N-terminal amino acids.

With wild-type cystatin C proteins, the amyloid formation phenomenon does not occur as readily under physiological conditions but human cystatin C with the L68Q mutation, dimerizes readily under physiological conditions. Thus, several mutations destabilize the monomeric structures, making it possible for the mutant proteins to form dimers readily by a “domain-swapping” mechanism.

“Domain-swapping” is a process in which a domain in a protein reorganizes its non-covalent bonding within the molecule and has similar bonding with the same domain of a second molecule. The theoretical consideration of domain-swapping in cystatins began with the studies of Ekiel and Abrahamson where they demonstrated that human cystatin C had the tendency to form inactive dimers under near-denaturing conditions.

For each of the cystatins, NMR chemical shift changes indicated that dimerization did not involve structural rearrangement of the main fold. However, rearrangements in the active site regions of the molecules were prevalent. The slow kinetics and high activation energy observed for dimerization reactions suggested intermolecular domain-swapping instead of just a simple association.

The level of cystatin C is greatly reduced in cerebrospinal fluid (CSF) of individuals suffering from Multiple Sclerosis (MS) in comparison with healthy controls. Reduced cystatin C in CSF of these patients was accompanied by an increased cysteine protease activity in the fluid compared with normal subjects. The reduced cysteine proteinase inhibition resulting from lesser presence of cystatin C is associated with if or if not itself a step in the pathogenesis of MS. A hypothetical role of cystatin C in MS is to modulate the activity levels of cathepsin B—and possibly other cysteine proteases—and may not be as a lead participant in the pathogenesis of the disease.

The level of cystatin C in the bloodstream is frequently assessed in relatively healthy individuals as a clinical marker of kidney function; raised levels are suggestive that the kidneys are not working optimally in protein resorption/retention. Cystatin C is freely filtered at the glomerulus. After filtration, cystatin C is reabsorbed and catabolized by the tubular epithelial cells, with only small amounts excreted in the urine. Cystatin C levels are therefore usually measured not in the urine, but in the bloodstream.

Cystatin C is but one component of integrated plaques. Cystatin C however, serves as a backbone, agglomeration seed or anchor, and model for multiplexed plaque deposits. Several claimed embodiments of this invention while directed specifically at the cystatin C molecule, by reducing this plaque instigation source reduce the number and size of growing multimeric agglomerations with the effect of slowing progression of plaque disease and also tilting the balance in favor of the body's plaque removal mechanisms.

The ideal marker for Glomerular Filtration Rate (GFR) would be produced and at a constant rate endogenously, regardless of age, sex, weight, and disease state. It would be filtered and excreted by the kidney only without renal tubular secretion and reabsorption, and once in urine it should be stable to allow for later analysis. Although creatinine meets some of these conditions, it has several limitations. Creatinine is produced in muscle. The amount produced therefore depends on muscle mass and is influenced by body type, age and gender. Creatine does not bind plasma proteins, and though it is freely filtered by the kidney, it is also secreted by the renal tubules. One serious drawback for creatinine as a marker for GFR is that it is not a sensitive marker of early GFR. This requires collection of timed urinary samples with application of a formula or algorithm that requires input data from measurement of both serum and urinary forms of creatinine. Also, rhabdomyolisis and/or eating uncooked meats can dramatically raise serum creatinine. This will skew the readings of urinary creatinine (one of the two sources of creatines measured for formula entry). Because of these drawbacks, several other endogenous markers for GFR have been evaluated. So far, the most promising marker which can potentially replace creatinine is our cystatin C.

Cystatin C has proven to be a suitable marker because it satisfies most of the above established criteria. Simonsen et al. first noted the excellent correlation between Cystatin C secretion and GFR when compared with exogenous markers of GFR, such as 51 Cr-EDTA. Cystatin C is present in high concentrations in serum, saliva, and seminal, synovial, and cerebrospinal fluids. It is produced and secreted at a constant rate by most nucleated cells and it is freely filtered by the glomerulus because of its small size. Unlike creatinine, serum cystatin C is not secreted by renal tubular epithelial cells, although they reabsorb and catabolize it. Secreted cystatin C therefore does not return to the bloodstream in its intact form.

The use of cystatin C as a renal function marker has produced familiarity with monitoring cystatin C levels in plasma and in urine. Handling, identifying and assaying amounts of the protein are thus commonplace with multiple vendors offering kits.

Ultimately in the diseased state, cystatin Cs aggregate to form proteinaceous deposits called amyloid β (Aβ) in various tissue areas. Amyloid is a generic term for an insoluble, fibrous protein deposit which can be formed by many different proteins. Amyloids are implicated in diseases such as dementia, stroke and brain hemorrhage.

In HCCAA, amyloid deposits are laid down in the blood vessels of the brain and other tissues. Troublesome symptoms including, but not limited to: brain hemorrhage, stroke, dementia and death often before the age of forty. The L68Q mutation involves inheritance of a gene that causes a leucine at position 68 to be replaced by a glutamine. This mutation results in a hydrophobic (non-polar) amino acid leucine, that normally resides in the heart of the folded protein where it avoids contact with water being replaced with a water loving glutamine. Residue 68 is buried deep within a hydrophobic core of the folded protein between the helix and the sheet structures. Placement of a polar (and slightly larger) glutamine at this position structurally destabilizes the monomeric protein. The molecule then is more likely to ‘flip out’ the inner portion as the normally hydrophobic core portion that holds the wild type molecule together is destabilized. In effect, the mutation makes the stable structure much more sensitive surface interactions and more likely to be sprung open exposing the polar glutamine to water and the flanking hydrophobic residues to an outside surface where they can contact and coordinate with other such hydrophobic regions. These interactions lead to dimers and higher aggregates that cause the amyloid linked pathologies.

Especially in the interstitial or intercellular micro-environment the cysteine proteases are inhibited by cystatins, particularly Cystatin C, which is secreted to extracellular space. Cysteine proteases share a common catalytic mechanism that comprises a nucleophilic cysteine thiol in a catalytic triad or dyad. Cysteine proteases are numerous and therefore are classified in accordance to their membership in one of the designated families assigned into 14 superfamilies: CA, CD, CE, CF, CL, CM, CN, CO, CP, PA, PB, PC, PD, PE, with several families (C7, C8, C21, C23, C27, C36, C42, C53 and C75) not fitting neatly into one of the superfamilies.

Preferred familial members of these superfamilies include but are not limited to: C1, C2, C3, C4, C5, C6, C9, C10, C11, C12, C13, C14, C15, C16, C18, C19, C25, C26, C28, C30, C31, C32, C33, C37, C39, C40, C44, C45, C46, C47, C48, C50, C51, C54, C55, C56, C57, C58, C59, C62, C63, C64, C65, C66, C67, C69, C70, C71, C74, C76, C78, C79, C80, C83, c84, C85, C86, C87, C89, C93, C95, C96, C97, C98, C99, C101 and P1.

Common examples of cysteine proteases include papain, bromelain, cathepsins B, C, F, H, K, L1, L2, O, S, W and Z (Cathepsin K is especially prevalent in extracellular space), calpain, caspace 1, separase, adenain, pyroglutamyl-peptidase 1, sortase A, hepatitis C virus, peptidase 2, sindbis virus-type nsP2 peptidase, dipeptidyl peptidase, TEV protease, hedgehog protein, amidophosphoribosyltransferase precursor, gamma-glutamyl hydrolase, DmpA aminopeptidase, etc. Human and non-human cysteine proteases have the cysteine thiol dyad or tryad in common and thus serve as binding ligands and/or inhibitors of cystatins. Cysteine proteases engineered to have diminished or absent proteolytic activity may be especially useful as therapeutic compounds, so long as the cystatin binding site is retained and accessible.

The source cysteine protease may be from any biologic source, including, but not limited to: viruses, archaebacteria, bacteria, plants, protists, fungi and mammals.

Cystatin C is a 13 kD non-glycosylated basic protein, that is often found to be elevated in diabetic patients even before the appearance of microalbuminuria. Cystatin C is used as a biologic marker for detecting nephropathy in patients with normoalbuminuria (early nephropathy). Serum and/or urinary levels of cystatin C are elevated in type 2 diabetics when compared to non-diabetic controls. A significant positive correlation was found between cystatin C levels and albuminuria.

The present invention provides methods for reducing the level of amyloid protein in a cell or tissue, the methods generally involving contacting the cell or tissue with an agent that reduces cystatin C levels and/or activity. Working with HCCAA patients has shown that while not a serious part of the amyloid disease processes, early amyloid agglomerations can be assayed using relatively non-invasive skin or mucous membrane biopsy. Dimers and dimer fragments are observable in blood. Early detection of amyloid disease presence and/or progression permits intervention to arrest, prevent, and possibly reverse amyloid associated disease before appreciable harm and symptoms are observable. The simple assays can be accomplished by the patient or an associate of the patient with no clinical experience required. Kits may be provided in a physician's office, pharmacy, remote dispensary, by mail, etc. that allow the patient to self-collect or have an associate collect the sample(s) for packaging, delivery and analysis. Another kit format may include near instantaneous readout similar to devices used by diabetics. For convenient shorthand, as used herein the term “posting” may include any mode of delivery, e.g., including using a professional delivery service, such as USPS, UPS, FedEx, etc., a local delivery service such as Lyft, Uber, taxi, etc., a friend, associate, relative, or neighbor, etc., or even dropped off in person. Results are preferably delivered electronically to at least one of the patient, relevant advisors, counselors, relatives, personal devices, etc.

Early stage treatments using glutathione support pathways and co-administered anti-amyloid disease interventions can thus commence previous to blockages and/or deterioration in any of the locations where amyloids have been associated with the various diseases. A co-administered substance is not required to be in the same form (IV and oral routes may co-exist) or even at the same time. The co-administered support substances may follow a different schedule for delivery which for multiple substances may be in series or parallel.

The present invention further provides methods for identifying an agent that reduces cystatin C levels and/or activity. One of several preferred embodiments includes maintaining and/or increasing availability and/or concentrations of glutathione or similar antioxidant, including, but not limited to: a glutathione delivering, synthesizing or maintaining effect. The presence or absence of reducing thiols (sulfhydryl groups that form the basis for antioxidant enzymes, glutathione, etc.) appears to directly affect cystatin C dimerization activities.

Glutathione is not stable when processed through gastro-enterological system, but can be delivered directly into the bloodstream, e.g., by iv drip, by intravenous injection or by inhalation. More preferred methods including, but not limited to: providing glutathione precursors, stimulating hepatic production with a corticosteroid compound like prednisolone or a synthetic analogue, biosimilar or like acting compound. Inhalation through, e.g., a nasal tube, a facemask, and/or a vapor and ambient air controlling tent may be a preferred administration route for mobility restricted, e.g., immobile, confined, or bedridden recipients.

One or more of several alternative or mutually supporting treatments having effects of increasing or maintaining glutathione levels are included as embodiments of the present invention. 8-bromo-cGMP provided at a rate of about 0.05 to 1 mg/kg/min, more preferably about 0.5 to 1.0 mg/kg/min for a time of about 1 min to 1 hr, more preferably, about 5 to 30 min can support glutathione levels. In glutathione depleted livers, S-adenosylmethionine (SAM) administration supports intrahepatic glutathione restoration to near-normal levels. SAM can be provided as an oral medicament, at a suggested dose of about 0.5 to 25 mg/kg with a range between about 5 to 10 mg/kg, commonly about 6 mg/kg or an approximate IM equivalent dose of about 0.2 to 10 mg/kg or commonly between about 2.5 mg·kg IM. Daily dosing of a single dose is often sufficient for effect, but dosing e.g., of from 1, 2, 3, 4, or 5 doses per day, especially with an oral medicament may be preferred.

N-acetylcysteine, cysteine esters, L-2-oxothiazolidine-4-carboxolate (“OTC”), gamma glutamylcysteine and its ethyl ester, glutathione ethyl ester, glutathione isopropyl ester, lipoic acid, cystine, cysteine, 3-morpholinosyndnonimine (SIN-1), methionine, and S-adenosylmethionine may be delivered to a patient who is expected to benefit from such glutathione augmentation treatment. Salts, nitrosylated, hydroxylated, esterified and similar pharmaceutically acceptable modifications are included in the above suggested compounds.

Dosages will be preferably from about 0.05 to about 500 mg/kg depending on the medicament and co-administered substance(s). E.g., vitamin C or vitamin E may be delivered preferentially at a dose of 1-20 mg/kg, e.g., about 50-1000 mg/day to a 50 kg (˜110 lb) person, about 100-2000 mg/day to a 100 kg person, about 150-3000 mg/day to a 150 kg person. Generally, a more preferred dosage will be about ⅓ the distance from the preferred minimum to the preferred maximum. Individual circumstances may result in a preferred dose for any individual falling slightly outside preferred ranges.

Glutathione precursors or pro-drugs capable of oral processing are preferred embodiments. E.g., γ-glutamylcysteine though poorly absorbed in free form is better absorbed when linked with protein such as whey. When taken with food or incorporated in a proteinaceous excipient such as yeast or yeast powder, or γ-glutamylcysteine becomes available as an immediate precursor for GSH synthase to convert to glutathione. γ-glutamylcysteine synthetase is inhibited by a glutathione feedback loop, but GSH synthase does not experience such negative feedback.

OTC at a dose of about 22-65 mg/kg can double cellular glutathione shortly after administration. However, its effect is short lived, waning in plasma by 8 hr post administration. Stronger and longer lasting effects were seen Another study with a dosage of 25 mg/kg OTC in conjunction with 800 mg/day glutathione IM found that OTC had continued effect as the glutathione was absorbed and distributed over time. Thus, OTA supplementation used alone to increase glutathione availability is preferably delivered at frequent doses, e.g., at least about 3 or more times per day or as an extended release or time released version for maximal continuous glutathione elevating effect. But OTC in conjunction with IM glutathione once per day has more prolonged effect that without the glutathione.

Vitamins B1, B2, B6, B9, B12, and selenium, magnesium, iron, and zinc metal ions or complexes, generally recommended for normal nutritional health, are preferably supplemented in prospective patients presenting with one or more deficiencies thereof.

Phytocannabinoid compounds including, but not limited to those present in: cinnamon, cumin, tumeric, ginger, cardamom, etc., may augment effectiveness of treatment.

The present invention also includes an inventive method for producing a medicament where the practitioner selects a person or population exhibiting a characteristic correlated with a disease diagnosis of interest. A biosample is obtained from the person or one or more members of the population so that a trait correlating with the disease can be measured in the biosample. To ensure the potential medicament is appropriate for the intended use, the potential medicament is administered to the person or at least a member of the population and the trait is remeasured following administration to determine effect of the potential medicament. If results are satisfactory the medicament can then be produced for use. Multiple potential medicaments may be considered and compared as above to select the optimal medicament for the person, population or sub-population.

The present invention provides methods leading to reducing the level of amyloid in a cell or a tissue. The methods generally involve contacting the cell or tissue with a substance that selectively reduces cystatin C dimerization in the cell or tissue. Reducing amyloid deposition is hypothesized to be effective for treating Alzheimer's disease (AD) in an individual. Thus, the present invention further provides methods of treating AD in an individual, the methods generally involving administering to an individual having AD a therapeutically effective amount of an agent determined to selectively reduce cystatin C levels and/or dimerization in the cell or tissue. The methods are also useful for treating cerebral amyloid angiopathy, cerebral hemorrhages, and generally dementia, in an individual.

When selecting a preferred substance, the selector will consider biological and chemical characteristics, such as mode of delivery as a therapeutic substance, specific tissues if any to be targeted, requirements for safe manufacture, consideration of positive and negative side effects, blood-brain barrier characteristics, potential for abuse, etc.

### Methods for Reducing Amyloid Levels

The present invention provides medicaments and methods of optimizing delivery of said medicaments to a patient standing to benefit therefrom. The medicament(s) leading to reduction of the level of amyloid deposit in a cell or a tissue, preferably in a cell or tissue of a live animal, more preferably in a cell or tissue of a live human. The methods generally involve contacting the cell or tissue with an agent that selectively reduces cystatin C level and/or amyloid deposits with a result of a reduced level of amyloid protein in the cell or tissue in comparison to the pre-medicament level or in comparison with at least one valid control.

Deposition of amyloids in the central nervous system is a recognized cause of decreased neuro-functionality. For treating these amyloids, therapeutics may be designed to cross the blood-brain barrier. Plaques within the circulatory system can be managed without necessity of blood-brain barrier crossing. For some mechanisms of the invention, a substance that can cross the blood brain barrier to arrest, slow or cause removal of amyloids or amyloid precursors is a preferred substance.

Substances that selectively reduce cystatin C levels and/or dimerization/oligomerization activity include, but are not limited to, an interfering nucleic acid that reduces the level of cystatin C expression in a cell or tissue; a mutant of cystatin C, a dominant mutant of cystatin C; a small molecule inhibitor of cystatin C dimerization, a small biologic derived substance that interferes with cystatin C interaction with other biologics including proteins such as cysteine proteases; an antibody, antibody fragment or modified antibody that specifically binds cystatin C and reduces binding of cystatin C, including dimerization and binding to cysteine proteases, the small biologic derived substance including, but not limited to: natural biologic molecules (including a biologic molecule secreted by a synthetic or genetically modified organism), a purified biologic substance, a biologic substance modified to alter activity, a biologic substance modified to modify its metabolism, a substance comprising a nucleic acid, a substance comprising a polypeptide, a substance comprising altered lipids, a substance comprising a lipoprotein, a substance with modified glycosylation, a substance modified to more easily cross the blood brain barrier, etc.

Effective substances to reduce cystatin C interactions may include glutathione precursors, e.g., a thiol donor including, but not limited to: L-cysteine and N-acetylcysteine and analogues and metabolic precursors thereof. Substances having antioxidant characteristics may also have desired qualities. The effective substance may be incorporated into an improved medicament preparation that might sustain length of activity, target a selected tissue or location, and/or protect from digestive or other metabolic events. For example, esters are sometimes suitable precursor molecules, and encapsulating substances—such as polymers—can delay metabolism of and/or protect the medicament from degradation to allow absorption through the intestinal wall.

N-acetylcysteine has been accepted and approved for human use. For example, acetaminophen toxicity has been ameliorated with n-acetylcysteine orally or IV at a dose approximating 140 mg/kg as a prime dose followed by 70 mg/kg doses each 4 hr period to achieve a total treatment of about 1300-1400 mg/kg over a three day period. N-acetylcysteine has been incorporated in skin patches (600 mg) with nitroglycerin for use thrice daily when treating unstable angina. Chronic bronchitis has been treated with n-acetylcysteine using various deliveries, including slow release and controlled release forms, totaling 400-600 mg/day. 600 mg/day is used as a COPD treatment or treatment adjunct. Elevated homocysteine is managed using doses up to about 1200 mg/day. Relatively high doses of n-acetylcysteine have been used to protect the bladder from isosfamide side effects using doses ranging from 800-8000 mg/day. And similarly high doses—4000-6000 mg/day have been used to treat myoclonus epilepsy. Doses are often expressed in base 2 or base 10 multiples as approximations of intended dosing. Patient mass may be measured in incremental single pounds or kilograms to determine actual dose, but more often doses are approximated using multiples of at least five, ten, twenty-five or even fifty. Thus the “about” used to describe approximate dosing includes a wide range commensurate with the loose approximations used for calculation.

These various safe, effective and accepted applications of n-acetylcysteine for multiple human interventions demonstrate safety and a degree of patient acceptance.

### ILLUSTRATIVE EXAMPLES

There are several hundred patients in Iceland who suffer from HCCAA (i.e., suffering major strokes in their early 20s) and they all descend from a founder mutation from the early 1500. The inventors have performed RNAseq on 30 subjects from 3 multiplex families and have shown that genes involved in coronary disease, stroke and atherosclerosis are upregulated in mutation carriers of cystatin C. To date there is no therapy available for these patients. Accordingly, intervention that has a potential to delay or reverse the disease process would be readily approved by the Icelandic Medicinal Agency.

Amyloid fiber dimerization is a critical step in the amyloid deposition process into small-medium sized brain arteries. In cell based assays, we have shown that both the wild type and mutated proteins are expressed and that expression of the mutated protein dimerizes, a process that can be inhibited. Thus, drugs that test as effective in an assay as blocking dimerization of the amyloid fibers are anticipated to be effective in preventing amyloid deposition and therefore to halt or reverse progression of the disease process. Such, selecting, analyzing, improving and reselecting, when successful, should, present increasingly effective therapies for HCCAA, and if robustly successful, e.g., in accessing the central nervous system tissue side of the blood brain barrier will become an effective therapy for AD.

In several embodiments biomolecules are elements of effective therapeutics. The natural biomolecules, when described or named are intended as shorthand expression to include, for example, a complete transcribed gene translated into a protein, splice variants, autolytic and other fragments, precursors, synthetic molecules including those comprising fragments including—when effective—fragments comprising D-amino acids and/or amino acids not having a natural codon in mammals, fragments or full length molecules comprised in a larger molecule, biosimilar substances, dimers and multimers of the original active ingredient with the active site accessible or accessible following normal metabolic processing, and prodrugs. Derivatives and prosubstances include but are not limited to: substances that metabolize to form the base substance, metabolites of the base substance maintaining at least one desired bioactivity, salt, hydroxides, hydroxylated, esters, etc. thereof.

This example of the invention commences with:
a) Selecting a substance capable of binding a cystatin C. Several such substances are known in the art including: i) papain and other members of the cysteine protease family such as falcipains; ii) glutathione, important for scavenging lipid hydroperoxides and reducing hydrogen peroxide iii) cathepsin B, a lysosomal cysteine protease; iv) selenium, a semi-metal atom in the oxygen sulfur family, with known toxicity but also important biologically in anti-oxidation though its presence in glutathione peroxidase; and v) monensin, an ionophoric anti-biotic that can facilitate passage of ions across cell membranes. Other substances with similar activity to the above might be selected depending on accessibility and/or additional substances might be conceived, synthesized or otherwise obtained for testing. Engineered analogues or chemical mimics of such biologics, including fragment and biosimilars and those engineered to no longer have the original bioactivity, but still retaining an available cystatin C binding site are included in this grouping.
b) Administering said substance to a cell culture or a cell culture extract preparation. Specific reaction conditions may be assessed here. Cell extract or synthetic cytoplasm like preparations might be used. For simplicity, to minimize screening costs, and/or to a balanced salt solution could be used as a viable substitute. Cells in culture are common vehicles for carrying out these type assessments. Monitoring results of said administering. Generally monitoring these types of results will require a control for comparison. The control may be a similar pharmaceutical substance, for example, an optical isomer. It may be a series of concentrations as common in making dose response curves. Dimerization itself can be monitored, alternatively, a surrogate or easily measured result with high correlation to dimerization can be measured.
c) Repeating a) through c) at least one time. Repeating may be coincidental (parallel) or repeated monitoring may be in subsequent experimental runs (serial). The repeating may also have an element of a control. Multiple results under different conditions, for example different concentrations or substances, will assist refinement of processes and choosing an optimal substance for therapeutic use.
d) Comparing results obtained in d). The plural result sets under different conditions facilitate selection, which may involve one or more of several factors, such as cost, efficacy, availability, durability (shelf-life), toxicity, partitioning to particular cells, tissues or organs, ability to cross membranes, ability to survive digestion, ability to cross the blood-brain barrier, customer acceptance, side effects, required dose, required number or frequency of dosing, regulatory body approval or clearance, reliability of supply chains, storage requirements, shipping restrictions, etc.
e) Selecting a preferred substance from results e). Based on factors mentioned above or other factors considered relevant to the patient or patient pool, a substance is selected as a preferred substance. A plurality of preferred substances may be selected if a plurality of conditions or patient types is considered.
f) And, providing said preferred substance for human use. The desire of the inventor is to improve the health, lifespan, or quality of life of at least one person. Preferably the invention will provide benefits to a large human population.

The invention may further comprise monitoring use by humans of the preferred substance, assessing safety, in a select population, expanding use to a larger population and monitoring members of said larger population for improved health or life conditions, especially improvement in a condition related to an associated disease.

Papain is marketed and used as a food supplement with dosing about 20-60 mg/day and suggested for treating swelling pain and inflammation at a dosage about 1500 mg/day. For example, twice daily or even about 3, 4, 5 or 6, even about 8, 9, 10 or more daily doses if taken as a candy or snack like popper, chew gum, or more, for example as a pill, etc., of about 5, 10, 20, 25, 30, 40, 50, 75, 100, 125, 150, 200, 250, 300, 500, 750, 1000 or more mg may be used.

Falcipains, malarial sourced cysteine proteases, have garnered interest medically as targets for fighting malaria. For example, a falcipain inhibitor is used to interfere with the malarial parasite's access to food. But, the enzymes can be put to use in an analogous manner to that of papain.

Cathepsin B has been used to induce apoptosis in several mammalian cells. It has also seen use as a dietary supplement to aid digestion. In ischemic injury cases inhibiting cathepsin B prevented significant loss of neurons in non-human primates and rodents. According to Wikipedia: in a transgenic mouse model for AD, inhibiting cathepsin B activity lessened memory deficit. But mice with higher levels of cathepsin B and human students performed better on memory tests. In these studies, cathepsin B was experimentally elevated. http://www.npr.org/sections/heaIth-shots/2016/06/23/483245084/a-protein-thatmoves-from-muscle-to-brain-may-tie-exercise-to-memory

Monensin is used as an ionophoric antibiotic supplement for animal feed up to about 700 g per day. Since it has preference for monovalent ions, including H⁺, monensin alters pH and has noted effect on the golgi affecting terminal glycosylation and proteolytic cleavages. Monovalent ions whose cotransport (neutral exchange, e.g., Na⁺/H⁺ exchange, across plasma membranes) have been studied include sodium, potassium, lithium, rubidium, titanium and silver. Dosages can be selected as tolerated and deemed effective. Suggested dosages that will be expected to be patient dependent range from about 50 to 500 mg per day with typical dosage 50 to 200 mg twice daily. Monensin can be tolerated with food typically from 1 to 4 times per day as low as about 20 mg per meal up to about 100 mg multiple times per day. Monensin is popular for veterinary applications in meat and poultry production especially effective in controlling intracellular parasites. The therapeutic window is relatively narrow compared to most human drugs. Monensin also affects intracellular ion transport and related functions including intracellular protein transport and the H⁺ gradient responsible for ATP production. Beneficial effects from monensin with respect to anti-agglomeration may include inducing antioxidant protein system expression in the affected cells. Skin biopsies, plasma and/or other fluid or tissue assays, including, but not limited to: saliva, tears, urine renal, hepatic, pulmonary, muscle, etc. may be used to determine levels of effectiveness.

Glutathione is found in many tissues, being naturally produced primarily in the liver as an antioxidant. Glutathione has been used therapeutically to treat cataracts, glaucoma, asthma, memory loss, AD, Parkinson's etc. http://www.webmd.com/vitamins-supplements/ingredientmono-717-GLUTATHIONE.aspx?activelngredientId=717&activelngredientName=GLUTATHIONE

A dose of 400-500 mg per day has been recommended as a supplement. Glutathione levels restored by a selenoprotein glutathione reductase.

The example continues by selecting one or a plurality of substances having characteristics similar to those of the above listed substances. Other proteases may be tested or other anti-oxidants may be included individually or as supplements in experimental protocols. The proteases may act as “sinks”, i.e., competitor molecules that distract the protease inhibitor from its usual target. Smaller molecules having the inhibitor binding domain, but not full enzymatic activity may be simpler to produce or administer and may have acceptable or even enhanced activity when compared to natural protease. Modified proteases that bind an inhibitor and then disrupt folding or cleave the inhibitor may be especially effective.

For this example, monensin, papain, a falcipain, and glutathione are the treatment cohorts with each given at three dosage levels. For preliminary studies 5 persons are at each dose for a total of 60 persons in the study.

Treatment may be guided in accordance with bioassays or testing protocols. Acceptance of a subject for treatment may be dependent on observations relating to symptoms and/or assays of one or more tissues or tissue samples. For example, genetic assays may be conducted to identify individuals at high risk for developing amyloidoses. For example, a mutation such as that related to L68Q cystatin C may be set as one simple threshold suggestive of treatment. Actual protein and/or plaque presence might also be set as a criterion for treatment. Treatment can be adjusted and repeated in accordance with a monitoring of disease progression or regression. Monitoring may precede and/or follow a treatment and may be repeated at random or defined intervals, for example, about seven days, about two weeks, about 30 days, about 45 days, about 60 days, about 90 days, about 120 days, about 180 days, about 360 days, and/or the like.

Monitoring can include imaging, functionality, bioassays, etc. Bioassay sources include but are not limited to: an integumentary biosample, a skin biosample, a buccal biosample, a mucus biosample, a blood biosample, a urinary biosample, a stool biosample, a muscle biosample, etc.

Such assaying may include at least one bioassay capable of determining amyloid deposit presence, cystatin C or fragment deposition, etc. and may involve a simple determination of the presence of the assayed substance or may involve quantitation and/or structure/size determination or the like.

Results of such monitoring events may be input into an algorithm, which may output a suggestion including one selected from but not limited to the group consisting of: at least one glutathione support compound, a dose relating to at least one glutathione support compound, a rate of delivering relating to at least one glutathione support compound and a route for said delivering relating to at least one glutathione support compound, etc.

Glutathione support compounds that might be used include but are not limited to: a sulfur containing compound selected from the group consisting of: sulfhydryls, thioethers, thioacetals, thioamino acids, thioproteins and thioesters. Such compound might be n-acetylcysteine, 2-mercaptoethanol, cysteine, glutathione, thioalkylate, furan-2-ylmethanethiol, grapefruit mercaptan, 2-propene-1-thiol, thioalkylates, thioredoxin, glutaredoxin, thiazolidinediones, methionine, etc.

Treatment may benefit from combination therapies. For example, an anti-inflammatory program may be advantageous for allowing continued activity of the glutathione supportive therapeutic(s). Macrophage and/or activity may interfere with access of the primary anti-plaque substances to the plaques and may in fact induce protein expression that increases expression of toxic amyloid. An anti-inflammatory compound may be one or more cytokine active substance, biosimilars and derivatives thereof and/or may be a synthetic compound, e.g., a small molecule, that inhibits inflammation. Small molecule anti-inflammatories including, but not limited to: sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121, MEG (mercaptoethylguanidine), etc. may be used in conjunction with the glutathione supportive compositions. Natural biomolecules such as interleukin (IL)-1 receptor antagonist, IL-4, IL-6, IL-10, IL-11, IL-13, precursors, active fragments and/or derivatives, preferably derived from the recipient species may also be advantageously supplemented to assist in inflammation control. Specific cytokine receptors for IL-1, tumor necrosis factor-α, and IL-18 may also function as proinflammatory cytokine inhibitors. Anti-inflammation actions of these compounds, in addition to modulating tissue damage from inflammatory responses, when used as a component in several embodiments of the present invention, in many cases will result in increased effectiveness of the treatment. I.e., slowing of additional plaque deposition, increased resorption of existing plaques, and decreased opportunity for plaque precursor compounds to react.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

For an early test of effectiveness of glutathione supportive therapy in the treatment of HCCAA and similar disease involving plaque formation, human trials are planned using accepted and previously approved therapeutic interventions. Initial trials involve n-acetylcysteine based on its chemistry and historical acceptance in human therapies. Doses ranging from as low as 200 mg/day, but at least in the early trials, at higher levels to emphasize effect have been proposed with initial regulatory approvals in high risk HCCAA patients.

### Applied Example

Three persons who had been identified through characteristic HCCAA symptoms confirmed by genetic screen to express L68Q cystatin C agreed to participate in a preliminary trial with a prototype drug to support antioxidant and other effects of GSH. N-acetylcysteine was used as a GSH-support/anti-cystatin-C-polymerization archetypical drug for conceptual proof and support of the hypotheses supporting this invention. All three patients showed disease arrest. One patient fully complied with the prescribed protocol. Two patients were only partially compliant. The fully compliant patient presented with a 50% reduction in plaques observed on biopsy.

In the partially compliant subject/patients, one showed a significant reduction of aggregated protein previously observed. The remaining non-compliant patient nevertheless showed disease arrest and may see improvement with future participation.

Treatments are continuing, augmented by confirmatory/advisory biopsies. Biopsy intervals may be approximately weekly, but longer intervals are envisioned as sufficient to monitor effects and dosages and to encourage compliance in testing and treatment maintenance. For example, biopsy intervals may be weekly, biweekly, monthly, bimonthly, quarterly, semi-annual, annual, etc. Biopsy appointments may be used both for treatment/disease monitoring, but also to maintain patient attention and interaction. Medical advisory panels and insurance issues may also be considered in determining the frequency and type(s) of biopsies.

1. A method for treating amyloid deposit disease comprising delivering:
a) at least one glutathione support compound; and
b) at least one anti-inflammatory substance,
to a patient presenting with or presenting with at least one risk element of amyloid disease.

2. The method of claim 1 wherein said glutathione support compound comprises an antioxidant.

3. The method of claim 1 wherein said at least one glutathione support compound comprises at least one substance or prosubstance that binds to and inhibits activity of cystatin C.

4. The method of claim 1 wherein said at least one glutathione support compound comprises at least one active compound selected from the group consisting of: selenium, n-acetylcysteine, S-adenosylmethionine and an ionophore.

5. The method of claim 5 wherein said at least one substance or prosubstance that binds to and inhibits activity of cystatin C comprises at least one cysteine protease molecule or derivative that binds or becomes capable of binding cystatin C.

6. The method of claim 7 wherein said substance or prosubstance comprises at least one member of a superfamily selected from the group consisting of: CA, CD, CE, CF, CL, CM, CN, CO, CP, PA, PB, PC, PD, PE and biosimilars and derivatives thereof.

7. The method of claim 8 wherein said substance or prosubstance comprises at least one member of a family selected from the group consisting of: C7, C8, C21, C23, C27, C36, C42, C53, C75 and biosimilars and derivatives thereof.

8. The method of claim 8 wherein said substance or prosubstance comprises at least one member of a family selected from the group consisting of: C1, C2, C3, C4, C5, C6, C9, C10, C11, C12, C13, C14, C15, C16, C18, C19, C25, C26, C28, C30, C31, C32, C33, C37, C39, C40, C44, C45, C46, C47, C48, C50, C51, C54, C55, C56, C57, C58, C59, C62, C63, C64, C65, C66, C67, C69, C70, C71, C74, C76, C78, C79, C80, C83, C84, C85, C86, C87, C89, C93, C95, C96, C97, C98, C99, C101 and P1 and biosimilars and derivatives thereof.

9. The method of claim 1, wherein said at least one glutathione support compound is delivered as a prodrug.

10. The method of claim 1 wherein said anti-inflammatory compound is selected from the group consisting of: interleukin (IL)-1 receptor antagonist, IL-4, IL-6, IL-10, IL-11, IL-13, cytokine receptors for IL-1, tumor necrosis factor-α, IL-18 and derivatives and biosimilars thereof, sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121, and MEG (mercaptoethylguanidine).

11. A medicament for decreasing amyloid deposits in an animal, said medicament comprising:
a) at least one glutathione support compound; and
b) at least one anti-inflammatory substance.

12. The medicament of claim 11 wherein said anti-inflammatory compound is selected from the group consisting of: a cytokine active substance, biosimilars and derivatives thereof and a synthetic compound that inhibits inflammation.

13. The medicament of claim 11 wherein said anti-inflammatory compound is selected from the group consisting of: interleukin (IL)-1 receptor antagonist, IL-4, IL-6, IL-10, IL-11, IL-13, cytokine receptors for IL-1, tumor necrosis factor-α, IL-18 and derivatives and biosimilars thereof, sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121, and MEG (mercaptoethylguanidine).

14. The medicament of claim 11 wherein said glutathione support compound is selected from the group consisting of: selenium, n-acetylcysteine, S-adenosylmethionine, 2-mercaptoethanol, cysteine, glutathione, thioalkylates, furan-2-ylmethanethiol, grapefruit mercaptan, 2-propene-1-thiol, thioredoxin, glutaredoxin, thiazolidinediones and methionine.

15. The medicament of claim 11 wherein said glutathione support compound is selected from the group consisting of: member of a superfamily selected from the group consisting of: CA, CD, CE, CF, CL, CM, CN, CO, CP, PA, PB, PC, PD, PE and biosimilars and derivatives thereof.

16. The medicament of claim 67 wherein said glutathione support compound comprises at least one member of a family selected from the group consisting of: C1, C2, C3, C4, C5, C6, C9, C10, C11, C12, C13, C14, C15, C16, C18, C19, C25, C26, C28, C30, C31, C32, C33, C37, C39, C40, C44, C45, C46, C47, C48, C50, C51, C54, C55, C56, C57, C58, C59, C62, C63, C64, C65, C66, C67, C69, C70, C71, C74, C76, C78, C79, C80, C83, C84, C85, C86, C87, C89, C93, C95, C96, C97, C98, C99, C101, C7, C8, C21, C23, C27, C36, C42, C53, C75, P1 and biosimilars and derivatives thereof.

17. The medicament of claim 14 comprising at least one compound selected from the group consisting of: interleukin (IL)-1 receptor antagonist, IL-4, IL-6, IL-10, IL-11, IL-13, cytokine receptors for IL-1, tumor necrosis factor-α, IL-18 and derivatives and biosimilars thereof and at least one compound selected from the group consisting of: sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121, and MEG (mercaptoethylguanidine).

18. The medicament of claim 11 comprising at least one supplement selected from the group consisting of: B1, B2, B6, B9, B12, and: selenium, magnesium, iron, and zinc metal complexes or ions.

19. The medicament of claim 11 comprising at least one supplement selected from the group consisting of: cinnamon, cumin, tumeric, ginger, cardamom, and other cannabinoid compounds having similar metabolic activities.

20. The method of claim 1 wherein said at least one risk element comprises a mutated cystatin C.

21. The method of claim 20 wherein said mutated cystatin C comprises a L68Q cystatin C.
